(19) United States
(12) Patent Application Publication
(10) Pub. No.: US 2026/0354919 A1
(43) Pub. Date: Nov. 12, 2026

(54) APPARATUS FOR ATTACHING CONTINUUM ROBOT TO ACTUATOR AND METHODS FOR USE THEREOF

(71) Applicant: CANON U.S.A., INC., MELVILLE, NY (US)

(72) Inventors: Charles George Hwang, Wellesley, MA (US); Inderpal Singh, Norwood, MA (US); Zachary Hamilton Haubert, Somerville, MA (US)

(21) Appl. No.: 19/203,846

(22) Filed: May 9, 2025

(51) Int. Cl. A61B 34/00; A61B 34/30; A61M 25/01; B25J 9/06; B25J 9/12

(52) U.S. Cl. CPC A61B 34/71; B25J 9/065; B25J 9/12; A61B 2034/301; A61M 25/0113; A61M 25/0147

(57) ABSTRACT

Disclosed are apparatuses and methods for safe, accurate, and expedited connection of a hub to an actuator for operation of a continuum robot to perform inspection, repair and/or medical procedures, including robotic medical procedures, with guide tools and devices that may include catheters, imaging devices and the like.

### BACKGROUND

### Field

The present disclosure relates generally to devices and methods for safe and expedited connection of a hub to an actuator for operation of a continuum robot to perform inspection, repair and/or medical procedures, including robotic medical procedures, with guide tools and devices that may include catheters, imaging devices and the like.

### Related Art

Flexible medical instruments such as endoscopic surgical devices and catheters are broadly used in surgical and probative settings. Such flexible medical instruments continue to gain acceptance in the medical field. The medical devices generally include a flexible tube commonly referred to as a sleeve or sheath, with one or more tool channels extending along or inside the sheath to allow access to end effectors located at a distal end of the sheath.

Conventional systems include U.S. Pub. 2021/0259794 to Kato et al., discussing medical apparatus with dual manipulation means, U.S. Pat. No. 11,559,190 to Okumura et al., discussing a steerable medical device, and U.S. Pub. 2021/0369085 to Kato et al., discussing a reflow trapped anchors. EP 2589407B1 discusses a catheter system with an attachable catheter hub via rotation around the hub for attachment. U.S. Pat. No. 11,553,917 to Sgroi discusses a loading unit locking collar with rotational actuated release. JP6857283B2 discusses a handheld surface cleaning unit in which components are mated together via tab flexes into a slot to lock components, with push tab release. JP2023103865A and U.S. Pub. 2021/0121051 discuss a medical device and bendable unit. JP 2022115015 discusses a medical apparatus having a main body that includes a plurality of drive sources, and an operation unit movable between a fixed position and a detached position when a bendable unit is attached to a base station.

CN 217430663U discusses a mounting bracket that is manually removably secured to the support bracket with clamshell orientation assembly for capture of a catheter assembly. U.S. Pat. No. 10,987,491 discusses a removable robotic catheter system that mates with a robot system by aligning mating holes. In such conventional systems, disposable catheter actuation interface(s) are mated to an actuation mechanism in the robotic catheter system by user alignment of vertical protrusions and associated mating holes.

### SUMMARY

To overcome various shortcomings of conventional systems, the present disclosure provides a system that allows for expedited and accurate attachment of a hub of a catheter/continuum robot to an actuator. The attachment is provided along a longitudinal direction, and the system provides safeguards against damage to proximal ends of the drive, i.e. actuation, wires that may result from undesired movement in one or more directions that are substantially orthogonal to the longitudinal direction. The present disclosure also expedites user alignment of the hub with the actuator while minimizing damage resulting from misalignment and actuation wire collision.

An aspect of the present disclosure provides an actuator that includes at least one clamp configured to receive a drive wire extending from a proximal end of a hub of a continuum robot; and one of a shelf or a protrusion. The shelf or a protrusion extends along a longitudinal axis beyond a distal end of the at least one clamp. The shelf or a protrusion is configured to align the hub with the actuator. Sliding the hub along the shelf or the protrusion towards the actuator transitions from a partially engaged state to an engaged state.

Another aspect of the present disclosure provides an actuator that includes at least one clamp configured to receive a drive wire extending from a proximal end of a continuum robot; and one of a shelf or a protrusion. The shelf or a protrusion extends along a longitudinal axis beyond a distal end of the at least one clamp and is configured to align the continuum robot with the actuator. Sliding the proximal end of the continuum robot along the shelf or the protrusion towards the actuator transitions from a partially engaged state to an engaged state.

A further aspect of the present disclosure provides a system for attaching a continuum robot to an actuator, that includes the actuator, the continuum robot, and one of a shelf or a protrusion. The actuator includes at least one clamp configured to receive a drive wire extending from a proximal end of the continuum robot. The shelf or a protrusion extends along a longitudinal axis beyond a distal end of the at least one clamp. The shelf or a protrusion is configured to align the proximal end of the continuum robot with a distal end of the actuator. Sliding the proximal end of the continuum robot along the shelf or the protrusion towards the actuator transitions from a partially engaged state to an engaged state.

### BRIEF DESCRIPTION OF THE DRAWINGS

The accompanying drawings, which are incorporated herein and form part of the specification, illustrate various embodiments, objects, features, and advantages of the present disclosure.

FIG. 1 illustrates components of a continuum robot connected with an actuator.

FIG. 2 illustrates a continuum robot system, according to an embodiment.

FIG. 3 is an expanded view of the continuum robot system of FIG. 2.

FIGS. 4A to 4D illustrate a process of aligning the continuum robot for attaching to the actuator, according to an embodiment.

FIG. 5 is a side perspective view of the continuum robot connected with the actuator, according to a first embodiment.

FIG. 6 is a front perspective view of the continuum robot connected with the actuator, according to the first embodiment.

FIG. 7 is a rear perspective view of the continuum robot connected with the actuator, according to the first embodiment.

FIG. 8 is a front perspective view of a protrusion for self-aligning the continuum robot with the actuator, according to a second embodiment.

FIG. 9 is a rear perspective view of the protrusion for self-aligning the continuum robot with the actuator, according to the second embodiment.

### DETAILED DESCRIPTION OF EXEMPLARY EMBODIMENTS

Aspects of the present disclosure can be understood by reading the following detailed description in light of the accompanying figures. It is noted that, in accordance with standard practice, the various features of the drawings are not drawn to scale and do not represent actual components. Several details such as dimensions of the various features may be arbitrarily increased or reduced for ease of illustration. In addition, reference numerals, labels and/or letters are repeated in the various examples to depict similar components and/or functionality. This repetition is for the purpose of simplicity and clarity and does not in itself limit the various embodiments and/or configurations the same components discussed.

Before the various embodiments are described in further detail, it shall be understood that the present disclosure is not limited to any particular embodiment. It is also to be understood that the terminology used herein is for the purpose of describing exemplary embodiments only, and is not intended to be limiting. Embodiments of the present disclosure may have many applications within the fields of inspection, repair and/or medical procedures, including minimally invasive surgery (MIS).

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of introduced claim recitation(s) is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As used herein, the term “real-time” is meant to describe processes or events communicated, shown, presented, etc. substantially at the same time as those processes or events actually occur. Real time refers to a level of computer responsiveness that a user senses as sufficiently immediate or that enables the computer to keep up with some external process. For example, in computer technology, the term real-time refers to the actual time during which something takes place and the computer may at least partly process the data in real time (as it comes in). As another example, in signal processing, “real-time” processing relates to a system in which input data is processed within milliseconds so that it is available virtually immediately as feedback, e.g., in a missile guidance, an airline booking system, or the stock market real-time quotes (RTQs).

The present disclosure may relate to medical devices, and exemplifies embodiments of a continuum robot, including an endoscope, catheter, or medical continuum robot, with embodiments thereof described in terms of three-dimensional space. As used herein, the term position may refer to location of an object or a portion of an object in a three-dimensional space, e.g., three degrees of translational freedom along Cartesian X, Y, Z coordinates. The term orientation may refer to the rotational placement of an object or a portion of an object, i.e., three degrees of rotational freedom—e.g., roll, pitch, and yaw. The term posture may refer to the position of an object or a portion of an object in at least one degree of translational freedom and to the orientation of that object or portion of object in at least one degree of rotational freedom, up to six total degrees of freedom. The term shape may refer to a set of posture, positions, and/or orientations measured along the elongated body of the object.

As known in the field, the terms “proximal” and “distal” are used with reference to the manipulation of an end of an instrument extending from the user to an inspection, surgical, and/or diagnostic site. In this regard, the term “proximal” refers to the portion of the instrument that is closer to the user, and the term “distal” refers to the portion (tip) of the instrument further away from the user and closer to an inspection, surgical and/or diagnostic site. It will be further appreciated that, for convenience and clarity, spatial terms such as “vertical”, “horizontal”, “up”, and “down” may be used herein with respect to the drawings. However, instruments are used in many orientations and positions, and these terms are not intended to be limiting and/or absolute. In that regard, all directional references (e.g., upper, lower, upward, downward, left, tight, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure.

As used herein the terms continuum robot and catheter may be interchangeably used to generally refer to a flexible and thin tubular instrument, typically made of medical grade material, designed to be inserted through a narrow opening. Such narrow opening may be for inspections of machinery, e.g. manufacturing equipment or complex components such as aircraft parts, such as may be performed by borescopes, fiber optic scopes, and the like. To observe an anatomical bodily lumen (e.g., an airway or a vessel), the steerable catheter/steerable continuum robot may be used to perform a broad range of medical functions. When used as an endoscope, a selectively rigid/flexible medical instrument provided by the steerable catheter/steerable continuum robot may be guided by light provided by an optical probe to observe within a bodily cavity or organ. Medical procedures in which an endoscope is inserted through a natural opening may be referred to as endoscopy. Specialized endoscopes are generally named for intended use, e.g. bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal.

In the present disclosure, the terms optical fiber, fiber optic, or fiber may each refer to an elongated, flexible, light conducting waveguide capable of conducting light from one end to another end due to the effect known as total internal reflection. The terms light guiding component or waveguide may also refer to, or may have the functionality of, an optical fiber. The term fiber may refer to one or more light conducting fibers.

### <Continuum Robot System>

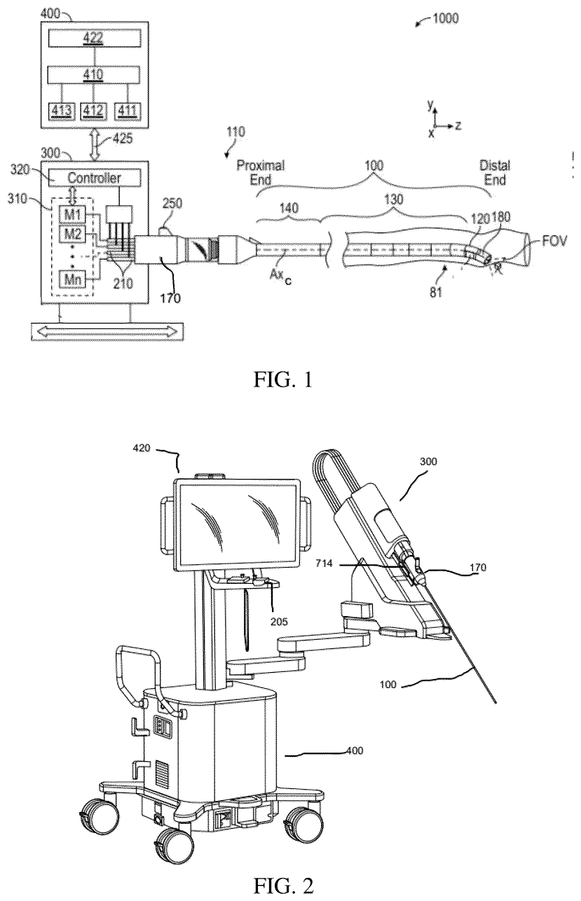

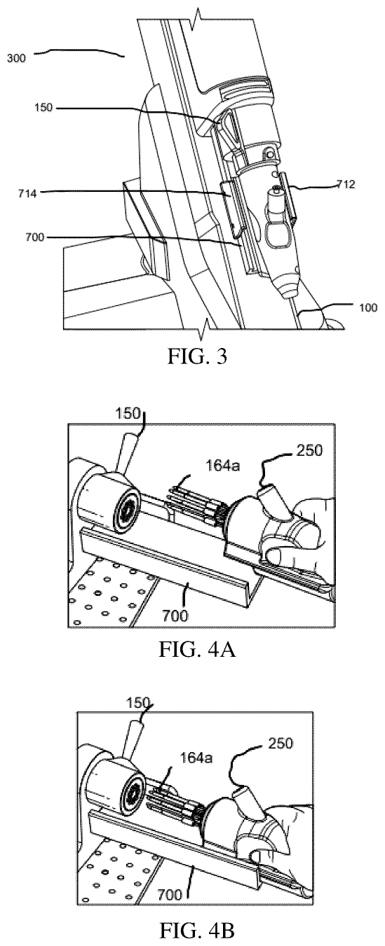

FIG. 1 illustrates components of a continuum robot connected with an actuator. FIG. 2 illustrates a continuum robot system, according to an embodiment. FIG. 3 is an expanded view of the continuum robot system of FIG. 2.

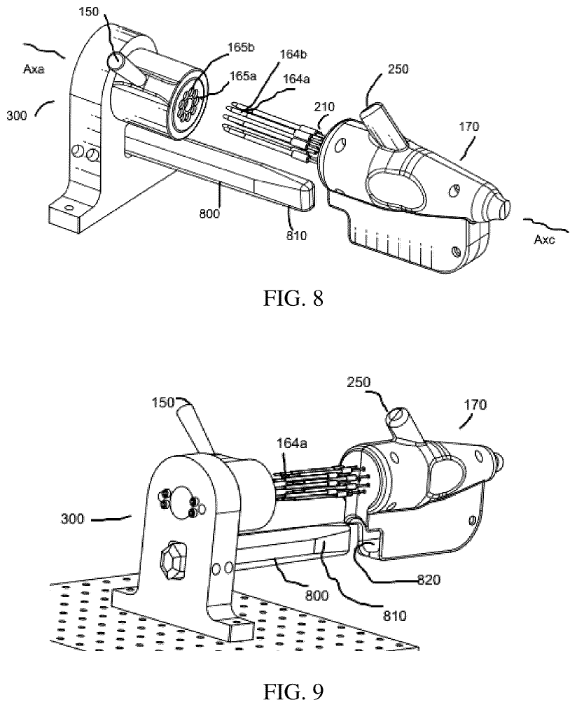

The continuum robot system 1000 may include an actuation handle or tab 150 provided at a distal end of the actuator 300. Rotation of the actuation handle 150 locks the continuum robot 100 to the actuator and operates at least two clamps to secure at least two attachment pins 164a, 164b to/from respective clamping mechanisms 165a, 165b (FIG. 8), also referred to a clamps, which are connected to respective tractors of the actuator 300. The continuum robot system 1000 may be used to perform inspections of pipes, lumens 81 and similar environs, as well as in an operating room or other medical environment to perform medical procedures, utilizing, inter alia, a field of view (FOV) provided from a distal end of the steerable continuum robot 100. The system 1000 may include a continuum robot/catheter 100 operable by a user to perform an inspection, an intraluminal procedure, and the like. The continuum robot/catheter 100 may include an elongated flexible shaft and at least one tool channel extending along or through a plurality of bendable segments for use in medical, repair and other processes. Imaging devices 180, tools and the like may be inserted into the tool channel view access port 250.

The steerable continuum robot 100 may include a proximal section 140, a distal section 130, and a rigid catheter tip 120, arranged in this order from the proximal end to the distal end along an axis (Axc) of the continuum robot. The distal section 130 includes a plurality of steerable bendable segments. The proximal section 140 includes a flexible non-steerable tubular shaft connecting the steerable section 130 to a hub 170.

The system 1000 may include a computer 400, e.g., system console, operatively connected to an actuator system 300. The computer 400 may include at least a central processing unit (CPU) 410 comprised of one or more than one processor, and a display screen 420 (FIG. 2) such as a liquid crystal display (LCD), OLED or QLED display. The CPU 410 may be operatively connected to a storage memory 411 (ROM and RAM memory), a system interface 412 (e.g., FPGA card), a user interface 413 (e.g. mouse and keyboard), and the display screen 420. The computer 400 may include suitable software, firmware, and peripheral hardware operated by one or more processors of CPU 410. The computer 400, the actuator system 300, and the actuation handle 150 are operably connected to each other by the network connection 425 (e.g., a cable bundle or wireless link).

The steerable continuum robot 100 may be controlled by an actuation system with a robotic platform and/or a handheld controller 205, e.g., a gamepad controller or joystick, which are in electronic communication with the computer 400 via the cable or network connection 425. The actuator system 300 may include a micro-controller 320 and actuator motors 310 which are operatively connected to the computer 400 via the network connection 425. The micro-controller 320 may include a proportional-integral-derivative (PID) controller or other similar digital signal processor (DSP) circuit. The actuator motors 310 may each include a respective actuating servo motors (piezoelectric actuators or tractors) M1 through Mn, with “n” being equal to a number of drive wires 210 for steering the steerable continuum robot 100 by changing a position and/or pose of the distal end. Each drive wire of the number of drive wires 210 extends from a proximal end of the hub 170, through the hub 170, to respective anchor points in the plurality of steerable bendable segments.

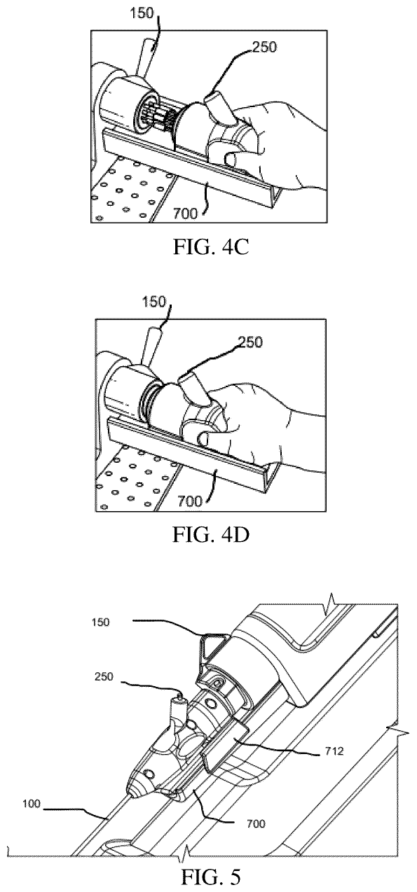

For attachment of the hub 170 to the actuator system 300, a user manually aligns the hub 170 to align axis Axc of the hub 170 with an axis Axa (FIG. 4A) of the actuator system 300. Axis Axc extends longitudinally through the hub 170. Axis Axa extends longitudinally through the actuator system 300.

While maintaining axis Axc in alignment with axis Axa, the user longitudinally moves the hub 170 towards the actuator system 300 to insert the proximal ends of the drive wires 210 into respective clamping mechanisms 165a,b of the actuator system 300.

Proper alignment of axis Axc with axis Axa provides correct orientation of proximal ends of the drive wires 210 with respective clamping mechanisms 165a,b of the actuator system 300.

Challenges arise when from misalignment of axis Axa and axis Axc. Such misalignment may readily occur as the user moves the hub 170 towards the actuator system 300.

In certain embodiments, nine control wires may extend from a proximal end of the hub 170 and the nine control wires may be symmetrically arranged around a circumference of the proximal end of the hub 170. As the user longitudinally moves the hub 170 towards the actuator system 300, the user may inadvertently misalign axis Axc and axis Axa. Lack of alignment or misalignment may arise from a change of pitch, and/or a change of yaw, and/or movement of the hub 170 relative to the actuator 300 in one or more of a sideways (Y) direction and/or a vertical (Z) direction.

Lack of alignment/misalignment may result in one or more proximal ends of the drive wires 210 not properly extending into respective clamping mechanisms 165a,b, i.e., creating an inaccurate fit. Inaccurate fit may result in clamping at non-uniform locations of the proximal ends of the drive wires 210. When the respective actuating servo motors (piezoelectric actuators/tractors) M1-Mn of the actuator 300 drive the respective drive wires, clamping at non-uniform locations may result in inaccurate bending of the distal section 130 and inaccurate steering of the steerable continuum robot 100.

When the system is assembled, as illustrated in FIGS. 1-3, a face of the hub 170 is configured to be flush fit against an opposing face of the actuator system 300. Misalignment may result in lack of the flush fit and undesired movement of the hub 170 may occur relative to the actuator system 300. Such undesired movement may also result in potentially inaccurate steering of the steerable continuum robot 100.

The present disclosure provides apparatuses, systems and methods that overcoming shortcomings of conventional systems by, inter alia, constraining movement of the hub 170 relative to the actuator system 300 in the Y and Z directions while permitting movement in the X direction, thereby ensuring proper alignment of the control wires with the corresponding holes for the respective clamping mechanisms 165a,b.

FIGS. 4A to 4D illustrate a process of aligning the continuum robot for attaching to the actuator, according to an embodiment.

As shown in FIGS. 4A to 4D, a shelf 700 extends longitudinally from a distal end of the actuator 300. Although the process described with reference to FIGS. 4A to 4D is based on use of the shelf 700, the process is not so limited and applies to each embodiment described herein.

In FIG. 4A, the actuator 300 and the hub 170 are fully disengaged from each other, in a non-contacting state. In the non-contacting state, the hub 170 may move in any one of the X, Y and/or Z directions relative to the actuator 300, without contacting shelf 700 or protrusion 800 (FIGS. 8-9).

The shelf 700 and/or protrusion 800 extend horizontally from the actuator 300 to provide a sliding surface perpendicular to the hub mating surface and allow distal ends of drive wires 200 of the continuum robot 100 that extend from a proximal end of the hub 170 to self-align with the corresponding holes in the actuator 300.

In FIG. 4B, a distal end of the shelf 700 contacts the proximal end of a hub 170, in an initial contacting state, also referred to as a partially engaged state. In the initial contacting state, downward movement (−Y direction) is precluded by the shelf 700, thereby facilitating vertical placement by a user of the hub 170 onto the shelf 700, i.e. placement from above.

A long at least a portion of the longitudinal axis, an upper portion of the shelf 700 has a first predetermined shape. As illustrated in FIGS. 4A-4D, the first predetermined shape may be concave. The first predetermined shape may also be one or more of a groove shape, a V shape, and/or a ridge shape, without being limited thereto.

A proximal portion of the hub (170) that is configured to slidably engage with the distal end of the shelf (700) has a second predetermined shape matching the first predetermined shape for self-alignment of the proximal portion of the hub 170 with the shelf 700. As illustrated in FIGS. 4A-4D, the second predetermined shape may be convex. The second predetermined shape may also be one or more of a groove shape, a V shape, and/or a ridge shape, without being limited thereto, to be configured to slidably fit to a respective first predetermined shape. As illustrated in FIGS. 4A-4D, the shelf 700 may have a concave shaped surface configured to mate with a convex surface of the bottom portion of the hub 170. When the bottom portion of the hub 170 is placed in the concave shaped surface portion of the shelf 700, sideward (+Y, −Y) movement is eliminated while allowing the hub 170 and continuum robot 100 to freely move in a longitudinal (+X, −X) direction, thus constraining the vertical and lateral movement during sliding. A small degree of movement in the vertical (−Z) direction may be obtained by pushing downward on the hub 170 as the hub 170 slides along the shelf 170 in the initial contacting state.

In the initial contacting state, the user aligns the hub 170 with the actuator 300 and then moves the hub 170, which is attached to the continuum robot 100, toward the actuator 300 in the X direction to advance to an engaged state (FIG. 4C). In the initial contacting state, movement is precluded in the downward (−Z) direction. Also in the initial contacting state, nesting of the second predetermined shape of the hub 170 into the first predetermined shape of the shelf 700 precludes certain lateral movement, i.e., in either sideward (+Y or −Y) direction.

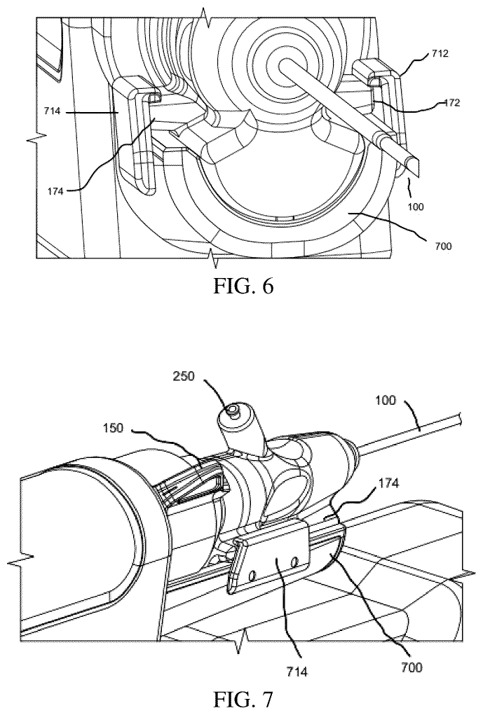

In FIG. 4C, the proximal end of a hub 170 has advanced longitudinally along the shelf 700 toward the actuator 300 to the engaged state. The longitudinal advancement occurs by sliding along a portion of a slidable surface of the shelf 700. In FIG. 4C, the proximal end of a hub 170 has longitudinally advanced from the initial contacting state along the shelf 700 toward the actuator 300 to a position where a port clip 712 (FIG. 6) and a starboard clip 714 contact opposite sides of the proximal end of a hub 170. In the engaged state, upward and downward (+Y and −Y) movement and sideward (+Z and −Z) movement is precluded by the shelf 700, the port clip 712, and the starboard clip 714. Thus, in the engaged state, movement of the hub 170 relative to the actuator 300 is precluded along the Y axis (sideward movement) and/or along the Z axis (vertical movement), while permitting movement along the X axis (longitudinal movement).

In FIG. 4D, the proximal end of a hub 170 has slid along an entire slidable surface of the shelf 700 to contact the actuator 300, to a connected state. The connected state is also illustrated in FIGS. 3 and 5-7. In the connected state, the actuation handle 150 locks the continuum robot 100 to the actuator, and movement of the hub 170 relative to the actuator 300 is precluded in each of the X, Y and Z directions.

Similar to the engaged state, in the connected state, movement of the hub 170 relative to the actuator 300 is precluded along the Y axis (sideward movement) and/or along the Z axis (vertical movement). However, in the connected state, movement is also precluded along the X axis (longitudinal movement).

Detachment of the hub 170 may be performed in the reverse order of the sequence described above in regard to FIGS. 4A to 4D.

### <Shelf System>

FIG. 5 is a side perspective view of the continuum robot connected with the actuator, according to a first embodiment. FIG. 6 is a front perspective view of the continuum robot connected with the actuator, according to the first embodiment. FIG. 7 is a rear perspective view of the continuum robot connected with the actuator, according to the first embodiment.

FIGS. 5-7 provide details of an embodiment of the port clip 712 and the starboard clip 714, in the connected state. In the connected state, as well as in the engaged state, movement of the actuator 300 is only permitted in the X direction.

As illustrated in FIGS. 5-7, the port clip 712 and the starboard clip 714 contact and pressingly engage against ridges that extend along opposite sides of the proximal end of the hub 170. The port clip 712 and the starboard clip 714 of the shelf 700 engage against the port ridge 172 and the starboard ridge 174, respectively, of the hub 170. The engaging of the clips against the ridges precludes movement other than in the X direction, to advance to/retreat from the engaged state, while maintaining self-alignment of the hub 170 with the shelf 700 and the actuator 300.

### <Protrusion System>

FIG. 8 is a front perspective view of a protrusion for self-aligning the continuum robot with the actuator, according to a second embodiment. FIG. 9 is rear perspective view of the protrusion for self-aligning the continuum robot with the actuator, according to the second embodiment.

Similar to the shelf 700 described with reference to FIGS. 2-7, the protrusion 800, also referred to as a rail, provides a surface perpendicular to the mating surface of the hub 170, for self-alignment of attachment pins 164a,b of the control wires 210 of the continuum robot 100 with corresponding clamping mechanisms 165a,b of the actuator 300.

As illustrated in FIG. 9, a hole 820 is provided to receive the protrusion 800. When the protrusion 800 is mated in the hole 820, at least one portions of the hub 170 surrounding a bottom portion of the hole 820 support a bottom portion of the protrusion 800. When the protrusion 800 is mated in the hole 820, movement is precluded in the Y and Z directions, while permitting movement in the X direction to advance to/retreat from the engaged state, and while maintaining self-alignment of the proximal portion of the hub 170 with the shelf 700.

As illustrated in FIGS. 8 and 9, a distal end of the protrusion 800 may include a taper 810 to facilitate mating of the protrusion 800 in the hole 820. In an alternative embodiment, the protrusion 800 may extend horizontally from the hub 170 and the hole may be formed in the actuator 300.

Thus, provided is an actuator that includes at least one clamp configured to receive a drive wire extending from a proximal end of a hub of a continuum robot and one of a shelf or a protrusion. The shelf or a protrusion extends along a longitudinal axis beyond a distal end of the at least one clamp. The shelf or a protrusion is configured to align the hub with the actuator. Sliding the hub along the shelf or the protrusion towards the actuator transitions from a partially engaged state to an engaged state. In the partially engaged state, the shelf or the protrusion restricts movement of the hub in at least one direction orthogonal to the longitudinal axis. Sliding of the hub along the shelf or the protrusion towards the actuator reduces a distance between a proximal end of the drive wire and the at least one clamp. The hub may move in a horizontal direction substantially parallel with the longitudinal axis when transitioning from the partially engaged state to the engaged state. In the engaged state, the shelf or the protrusion precludes movement of the hub, other than in the horizontal direction. In the partially engaged state or the engaged state, the hub cannot rotate relative to the actuator around the longitudinal axis.

The one of the shelf or the protrusion has a first length in the longitudinal direction. In the partially engaged state, a length from the proximal end of the drive wire to the proximal end of the hub is shorter than the first length.

The actuator may also include a plurality of drive wires arranged in a first predetermined pattern; and a plurality of clamps arranged in a second predetermined pattern. The first predetermined pattern matches the second predetermined pattern. During the sliding of the hub along the shelf or the protrusion towards the actuator, the plurality of drive wires remain in alignment with respective clamps of the plurality of clamps.

The shelf may include a first predetermined shape along an upper portion of the longitudinal axis. The hub may include a second predetermined shape along a lower portion of the longitudinal axis. The second predetermined shape matches the first predetermined shape.

Placing the hub on the shelf slidably engages the first predetermined shape with the second predetermined shape and aligns an attachment pin of the drive wire with the at least one clamp. One of the first predetermined shape or the second predetermined shape may be one of a concave shape, a groove shape, a V shape, and/or a ridge shape. The other one of the first predetermined shape or the second predetermined shape may be one of a convex shape, a groove shape, a V shape, and/or a ridge shape configured to slidably fit with a respective first predetermined shape.

The first predetermined shape may include a ridge extending from a distal end thereof along the longitudinal axis towards the actuator axis. The second predetermined shape may include a groove configured to slidably engage the ridge as the hub transitions from the partially engaged state to the engaged state. Engaging of the ridge in the groove precludes rotation of the hub relative to the actuator around the longitudinal axis, with the catheter being free to move in the x axis, which is desired. Movement in the z direction is controlled by lightly pushing down the catheter as it slides along the shelf.

The first predetermined shape may occupy less than or equal to 45 degrees of the upper portion of the shelf, and is arranged for user to place the hub on the shelf.

The protrusion may be configured to extend into an opening in the hub during the transition from the partially engaged state to the engaged state. The protrusion may have a first predetermined shape along at least a portion of the longitudinal axis. The opening in the hub may have a second predetermined shape matching the first predetermined shape. Engaging the first predetermined shape of the protrusion with the second predetermined shape precludes rotation of the hub relative to the actuator around the longitudinal axis. The distal end of the protrusion may include a taper.

The actuator may also include a clip extending vertically from an upper surface of the shelf. The clip may be positioned on an end of the shelf adjacent to the at least one clamp, and the clip may be configured to secure a ridge provided along the longitudinal axis of the hub.

An actuator is provided that includes at least one clamp configured to receive a drive wire extending from a proximal end of a continuum robot; and one of a shelf or a protrusion. The shelf or a protrusion may extend along a longitudinal axis beyond a distal end of the at least one clamp and may be configured to align the continuum robot with the actuator. Sliding the proximal end of the continuum robot along the shelf or the protrusion towards the actuator transitions from a partially engaged state to an engaged state. In the partially engaged state, the shelf or the protrusion restricts movement of the continuum robot in at least one direction orthogonal to the longitudinal axis. The continuum robot may move in a horizontal direction substantially parallel with the longitudinal axis when transitioning from the partially engaged state to the engaged state. In the engaged state, the shelf or the protrusion precludes movement of the continuum robot, other than in the horizontal direction.

The sliding along the shelf or the protrusion towards the actuator reduces a distance between a proximal end of the drive wire and the at least one clamp.

The one of the shelf or the protrusion has a first length in the longitudinal direction and, in the partially engaged state, a length from the proximal end of the drive wire to the proximal end of the continuum robot is shorter than the first length. During the transition from the partially engaged state to the engaged state, the protrusion is configured to extend into an opening in the continuum robot.

Also provided is a system for attaching a continuum robot to an actuator, the system including the actuator, the continuum robot, and one of a shelf or a protrusion. The actuator may include at least one clamp configured to receive a drive wire extending from a proximal end of the continuum robot. The shelf or a protrusion extends along a longitudinal axis beyond a distal end of the at least one clamp. The shelf or a protrusion may be configured to align the proximal end of the continuum robot with a distal end of the actuator. Sliding the proximal end of the continuum robot along the shelf or the protrusion towards the actuator transitions from a partially engaged state to an engaged state.

In the partially engaged state, the shelf or the protrusion restricts movement of the continuum robot relative to the actuator in at least one direction orthogonal to the longitudinal axis. The proximal end of the continuum robot moves in a horizontal direction substantially parallel with the longitudinal axis when transitioning from the partially engaged state to the engaged state and, in the engaged state, the shelf or the protrusion precludes movement of the continuum robot relative to the actuator, other than in the horizontal direction. The sliding of the proximal end of the continuum robot along the shelf or the protrusion towards the actuator reduces a distance between a proximal end of the drive wire and the at least one clamp.

The one of the shelf or the protrusion may have a first length in the longitudinal direction. In the partially engaged state, a length from the proximal end of the drive wire to the proximal end of the continuum robot is shorter than the first length.

The system may also include a plurality of drive wires arranged in a first predetermined pattern; and a plurality of clamps arranged in a second predetermined pattern. The first predetermined pattern matches the second predetermined pattern. During the sliding of the continuum robot along the shelf or the protrusion towards the actuator, the plurality of drive wires remain in alignment with respective clamps of the plurality of clamps.

The shelf may include a first predetermined shape along an upper portion of the longitudinal axis. The proximal end of the continuum robot may include a second predetermined shape along a lower portion of the longitudinal axis. The second predetermined shape matches the first predetermined shape. One of the first predetermined shape or the second predetermined shape may be one of a concave shape, a groove shape, a V shape, and/or a ridge shape. The other one of the first predetermined shape or the second predetermined shape may be one of a convex shape, a groove shape, a V shape, and/or a ridge shape configured to slidably fit with a respective first predetermined shape. The first predetermined shape may include a ridge extending from a distal end thereof along the longitudinal axis towards the actuator axis. The second predetermined shape may include a groove configured to slidably engage the ridge as the hub transitions from the partially engaged state to the engaged state. Engaging of the ridge in the groove precludes rotation of the continuum robot relative to the actuator around the longitudinal axis, with the catheter being free to move in the longitudinal direction.

The first predetermined shape may occupy less than or equal to 45 degrees of the upper portion of the shelf and may be arranged for user to place the proximal end of the continuum robot on the shelf. The protrusion may be configured to extend into an opening in the proximal end of the continuum robot during the transition from the partially engaged state to the engaged state. The protrusion may have a first predetermined shape along at least a portion of the longitudinal axis. The opening may have a second predetermined shape matching the first predetermined shape. Engaging the first predetermined shape of the protrusion with the second predetermined shape precludes rotation of the continuum robot relative to the actuator around the longitudinal axis.

The system may also include a clip extending vertically from an upper surface of the shelf. The clip may be positioned on an end of the shelf adjacent to the at least one clamp, and the clip may be configured to secure a ridge provided along the longitudinal axis of the proximal end of the continuum robot.

Thus, an aspect provides an actuator comprising at least one clamp configured to receive a drive wire extending from a proximal end of a hub of a continuum robot; and one of a shelf or a protrusion. The shelf or a protrusion extends along a longitudinal axis beyond a distal end of the at least one clamp. The shelf or a protrusion is configured to align the hub with the actuator. Sliding the hub along the shelf or the protrusion towards the actuator transitions from a partially engaged state to an engaged state.

In the partially engaged state, the shelf or the protrusion restricts movement of the hub in at least one direction orthogonal to the longitudinal axis.

The hub moves in a horizontal direction substantially parallel with the longitudinal axis when transitioning from the partially engaged state to the engaged state, and in the engaged state, the shelf or the protrusion precludes movement of the hub, other than in the horizontal direction.

The sliding of the hub along the shelf or the protrusion towards the actuator reduces a distance between a proximal end of the drive wire and the at least one clamp.

The one of the shelf or the protrusion has a first length in the longitudinal direction. In the partially engaged state, a length from the proximal end of the drive wire to the proximal end of the hub is shorter than the first length.

The actuator may further comprise a plurality of drive wires arranged in a first predetermined pattern, and a plurality of clamps arranged in a second predetermined pattern. The first predetermined pattern matches the second predetermined pattern. During the sliding of the hub along the shelf or the protrusion towards the actuator, the plurality of drive wires remain in alignment with respective clamps of the plurality of clamps.

The shelf may include a first predetermined shape along an upper portion of the longitudinal axis, the hub may include a second predetermined shape along a lower portion of the longitudinal axis, with the second predetermined shape matching the first predetermined shape.

Placing the hub on the shelf slidably engages the first predetermined shape with the second predetermined shape, and aligns an attachment pin of the drive wire with the at least one clamp.

One of the first predetermined shape or the second predetermined shape is one of a concave shape, a groove shape, a V shape, and/or a ridge shape, and the other one of the first predetermined shape or the second predetermined shape is one of a convex shape, a groove shape, a V shape, and/or a ridge shape configured to slidably fit with a respective first predetermined shape.

The first predetermined shape may include a ridge extending along the longitudinal axis, the second predetermined shape may include a groove configured to slidably engage the ridge as the hub transitions from the partially engaged state to the engaged state, and the engaging of the ridge in the groove precludes rotation of the hub.

The first predetermined shape occupies less than or equal to 45 degrees of the upper portion of the shelf, and is arranged for user to place the hub on the shelf.

The protrusion is configured to extend into an opening in the hub during the transition from the partially engaged state to the engaged state, with the protrusion having a first predetermined shape along at least a portion of the longitudinal axis, and the opening in the hub having a second predetermined shape matching the first predetermined shape. Engaging the first predetermined shape of the protrusion with the second predetermined shape precludes rotation of the hub. The distal end of the protrusion may include a taper.

The actuator may also comprise a clip extending vertically from an upper surface of the shelf, wherein the clip is positioned on an end of the shelf adjacent to the at least one clamp, and the clip is configured to secure a ridge provided along the longitudinal axis of the hub.

Another aspect of the present disclosure provides an actuator that comprises at least one clamp configured to receive a drive wire extending from a proximal end of a continuum robot; and one of a shelf or a protrusion. The shelf or a protrusion extends along a longitudinal axis beyond a distal end of the at least one clamp and is configured to align the continuum robot with the actuator. Sliding the proximal end of the continuum robot along the shelf or the protrusion towards the actuator transitions from a partially engaged state to an engaged state.

In the partially engaged state, the shelf or the protrusion restricts movement of the continuum robot in at least one direction orthogonal to the longitudinal axis; the continuum robot moves in a horizontal direction substantially parallel with the longitudinal axis when transitioning from the partially engaged state to the engaged state; and in the engaged state, the shelf or the protrusion precludes movement of the continuum robot, other than in the horizontal direction. The sliding along the shelf or the protrusion towards the actuator reduces a distance between a proximal end of the drive wire and the at least one clamp.

The one of the shelf or the protrusion has a first length in the longitudinal direction. In the partially engaged state, a length from the proximal end of the drive wire to the proximal end of the continuum robot is shorter than the first length.

During the transition from the partially engaged state to the engaged state, the protrusion is configured to extend into an opening in the continuum robot.

Another aspect of the present disclosure provides a system comprising an actuator; a continuum robot; and one of a shelf or a protrusion. The actuator includes at least one clamp configured to receive a drive wire extending from a proximal end of the continuum robot. The shelf or a protrusion extends along a longitudinal axis beyond a distal end of the at least one clamp. The shelf or a protrusion is configured to align the proximal end of the continuum robot with a distal end of the actuator. Sliding the proximal end of the continuum robot along the shelf or the protrusion towards the actuator transitions from a partially engaged state to an engaged state.

In the partially engaged state, the shelf or the protrusion restricts movement of the continuum robot relative to the actuator in at least one direction orthogonal to the longitudinal axis.

The proximal end of the continuum robot moves in a horizontal direction substantially parallel with the longitudinal axis when transitioning from the partially engaged state to the engaged state. In the engaged state, the shelf or the protrusion precludes movement of the continuum robot relative to the actuator, other than in the horizontal direction.

The sliding of the proximal end of the continuum robot along the shelf or the protrusion towards the actuator reduces a distance between a proximal end of the drive wire and the at least one clamp.

The one of the shelf or the protrusion has a first length in the longitudinal direction. In the partially engaged state, a length from the proximal end of the drive wire to the proximal end of the continuum robot is shorter than the first length.

The system may further comprise:
a plurality of drive wires arranged in a first predetermined pattern; and
a plurality of clamps arranged in a second predetermined pattern, wherein:
the first predetermined pattern matches the second predetermined pattern, and
during the sliding of the continuum robot along the shelf or the protrusion towards the actuator, the plurality of drive wires remain in alignment with respective clamps of the plurality of clamps.

The shelf may include a first predetermined shape along an upper portion of the longitudinal axis. The proximal end of the continuum robot may include a second predetermined shape along a lower portion of the longitudinal axis. The second predetermined shape matches the first predetermined shape. One of the first predetermined shape or the second predetermined shape is one of a concave shape, a groove shape, a V shape, and/or a ridge shape. The other one of the first predetermined shape or the second predetermined shape is one of a convex shape, a groove shape, a V shape, and/or a ridge shape configured to slidably fit with a respective first predetermined shape.

The first predetermined shape may include a ridge extending along the longitudinal axis. The second predetermined shape may include a groove configured to slidably engage the ridge as the hub transitions from the partially engaged state to the engaged state. The engaging of the ridge in the groove precludes rotation of the continuum robot relative to the actuator.

The first predetermined shape occupies less than or equal to 45 degrees of the upper portion of the shelf and is arranged for user to place the proximal end of the continuum robot on the shelf.

The protrusion is configured to extend into an opening in the proximal end of the continuum robot during the transition from the partially engaged state to the engaged state, the protrusion has a first predetermined shape along at least a portion of the longitudinal axis, the opening has a second predetermined shape matching the first predetermined shape, and engaging the first predetermined shape of the protrusion with the second predetermined shape precludes rotation of the continuum robot relative to the actuator.

The may further comprise a clip extending vertically from an upper surface of the shelf. The clip may be positioned on an end of the shelf adjacent to the at least one clamp. The clip may be configured to secure a ridge provided along the longitudinal axis of the proximal end of the continuum robot.

### REFERENCE NUMBERS

| | |
|---|---|
| 81 | lumen |
| 100 | continuum robot |
| 120 | catheter tip |
| 130 | distal section |
| 140 | proximal section |
| 150 | actuation handle |
| 164a, b | attachment pins |
| 165a, b | clamping mechanisms |
| 170 | hub |
| 172 | port ridge |
| 174 | starboard ridge |
| 180 | imaging device |
| 205 | handheld controller |
| 210 | drive wires |
| 250 | access port |
| 300 | actuator |
| 310 | actuator motors |
| 320 | micro-controller |
| 400 | computer |
| 410 | CPU |
| 411 | memory |
| 412 | system interface |
| 413 | user interface |
| 420 | display screen |
| 425 | network connection |
| 700 | shelf |
| 712 | port clip |
| 714 | starboard clip |
| 800 | protrusion |
| 820 | hole |
| 1000 | continuum robot system |

In referring to the above description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Each patent, patent publication and other publication identified herein and examples described herein are expressly incorporated herein by reference. These publications are provided solely for their disclosure prior to the filing date or applicable priority date of the present application. Nothing in this regard shall be construed as an admission against antedating such publication(s). All statements as to the date or representation as to content/disclosure of such publication(s) is based on information available and does not constitute any admission as to accuracy of date of disclosure/publication of such publication(s) or content of such publication(s).

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans are expected to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. An actuator comprising:
at least one clamp configured to receive a drive wire extending from a proximal end of a hub of a continuum robot; and
one of a shelf or a protrusion, wherein:
the shelf or a protrusion extends along a longitudinal axis beyond a distal end of the at least one clamp,
the shelf or a protrusion is configured to align the hub with the actuator, and
sliding the hub along the shelf or the protrusion towards the actuator transitions from a partially engaged state to an engaged state.

2. The actuator of claim 1, wherein, in the partially engaged state, the shelf or the protrusion restricts movement of the hub in at least one direction orthogonal to the longitudinal axis.

3. The actuator of claim 1, wherein,
the hub moves in a horizontal direction substantially parallel with the longitudinal axis when transitioning from the partially engaged state to the engaged state, and
in the engaged state, the shelf or the protrusion precludes movement of the hub, other than in the horizontal direction.

4. The actuator of claim 1, wherein, the sliding of the hub along the shelf or the protrusion towards the actuator reduces a distance between a proximal end of the drive wire and the at least one clamp.

5. The actuator of claim 1, wherein:
the one of the shelf or the protrusion has a first length in the longitudinal direction, and
in the partially engaged state, a length from the proximal end of the drive wire to the proximal end of the hub is shorter than the first length.

6. The actuator of claim 1, further comprising:
a plurality of drive wires arranged in a first predetermined pattern; and
a plurality of clamps arranged in a second predetermined pattern, wherein:
the first predetermined pattern matches the second predetermined pattern, and
during the sliding of the hub along the shelf or the protrusion towards the actuator, the plurality of drive wires remain in alignment with respective clamps of the plurality of clamps.

7. The actuator of claim 1, wherein:
the shelf includes a first predetermined shape along an upper portion of the longitudinal axis,
the hub includes a second predetermined shape along a lower portion of the longitudinal axis,
the second predetermined shape matches the first predetermined shape, and
placing the hub on the shelf slidably engages the first predetermined shape with the second predetermined shape, and aligns an attachment pin of the drive wire with the at least one clamp.

8. The actuator of claim 7, wherein:
one of the first predetermined shape or the second predetermined shape is one of a concave shape, a groove shape, a V shape, and/or a ridge shape, and
the other one of the first predetermined shape or the second predetermined shape is one of a convex shape, a groove shape, a V shape, and/or a ridge shape configured to slidably fit with a respective first predetermined shape.

9. The actuator of claim 7, wherein:
the first predetermined shape includes a ridge extending along the longitudinal axis,
the second predetermined shape includes a groove configured to slidably engage the ridge as the hub transitions from the partially engaged state to the engaged state, and
the engaging of the ridge in the groove precludes rotation of the hub.

10. The actuator of claim 7, wherein:
the first predetermined shape occupies less than or equal to 45 degrees of the upper portion of the shelf, and is arranged for user to place the hub on the shelf.

11. The actuator of claim 1, wherein:
the protrusion is configured to extend into an opening in the hub during the transition from the partially engaged state to the engaged state,
the protrusion has a first predetermined shape along at least a portion of the longitudinal axis,
the opening in the hub has a second predetermined shape matching the first predetermined shape, and
engaging the first predetermined shape of the protrusion with the second predetermined shape precludes rotation of the hub.

12. The actuator of claim 1, further comprising:
a clip extending vertically from an upper surface of the shelf, wherein:
the clip is positioned on an end of the shelf adjacent to the at least one clamp, and
the clip is configured to secure a ridge provided along the longitudinal axis of the hub.

13. An actuator comprising:
at least one clamp configured to receive a drive wire extending from a proximal end of a continuum robot; and
one of a shelf or a protrusion, wherein:
the shelf or a protrusion extends along a longitudinal axis beyond a distal end of the at least one clamp and is configured to align the continuum robot with the actuator,
sliding the proximal end of the continuum robot along the shelf or the protrusion towards the actuator transitions from a partially engaged state to an engaged state,
in the partially engaged state, the shelf or the protrusion restricts movement of the continuum robot in at least one direction orthogonal to the longitudinal axis,
the continuum robot moves in a horizontal direction substantially parallel with the longitudinal axis when transitioning from the partially engaged state to the engaged state, and
in the engaged state, the shelf or the protrusion precludes movement of the continuum robot, other than in the horizontal direction.

14. The actuator of claim 13, wherein:
the sliding along the shelf or the protrusion towards the actuator reduces a distance between a proximal end of the drive wire and the at least one clamp,
the one of the shelf or the protrusion has a first length in the longitudinal direction, and
in the partially engaged state, a length from the proximal end of the drive wire to the proximal end of the continuum robot is shorter than the first length.

15. A system comprising:
an actuator;
a continuum robot; and
one of a shelf or a protrusion extending in a horizontal direction, wherein:
the actuator includes at least one clamp configured to receive a drive wire extending from a proximal end of the continuum robot, and
the shelf or a protrusion extends along a longitudinal axis beyond a distal end of the at least one clamp,
the shelf or a protrusion is configured to align the proximal end of the continuum robot with a distal end of the actuator, and
sliding the proximal end of the continuum robot along the shelf or the protrusion towards the actuator transitions from a partially engaged state to an engaged state.

16. The system of claim 15, wherein:
in the partially engaged state, the shelf or the protrusion restricts movement of the continuum robot relative to the actuator in at least one direction orthogonal to the longitudinal axis,
the proximal end of the continuum robot moves in the horizontal direction substantially parallel with the longitudinal axis when transitioning from the partially engaged state to the engaged state,
in the engaged state, the shelf or the protrusion precludes movement of the continuum robot relative to the actuator, other than in the horizontal direction, and
the sliding of the proximal end of the continuum robot along the shelf or the protrusion towards the actuator reduces a distance between a proximal end of the drive wire and the at least one clamp.

17. The system of claim 15, wherein:
the one of the shelf or the protrusion has a first length in the longitudinal direction, and
in the partially engaged state, a length from the proximal end of the drive wire to the proximal end of the continuum robot is shorter than the first length.

18. The system of claim 15, further comprising:
a plurality of drive wires arranged in a first predetermined pattern; and
a plurality of clamps arranged in a second predetermined pattern, wherein:
the first predetermined pattern matches the second predetermined pattern, and
during the sliding of the continuum robot along the shelf or the protrusion towards the actuator, the plurality of drive wires remain in alignment with respective clamps of the plurality of clamps.

19. The system of claim 15, wherein:
the shelf includes a first predetermined shape along an upper portion of the longitudinal axis,
the proximal end of the continuum robot includes a second predetermined shape along a lower portion of the longitudinal axis,
the second predetermined shape matches the first predetermined shape,
one of the first predetermined shape or the second predetermined shape is one of a concave shape, a groove shape, a V shape, and/or a ridge shape,
the other one of the first predetermined shape or the second predetermined shape is one of a convex shape, a groove shape, a V shape, and/or a ridge shape configured to slidably fit with a respective first predetermined shape, and
the first predetermined shape occupies less than or equal to 45 degrees of the upper portion of the shelf, and is arranged for user to place the proximal end of the continuum robot on the shelf.

20. The system of claim 15, further comprising:
a clip extending vertically from an upper surface of the shelf, wherein:
the clip is positioned on an end of the shelf adjacent to the at least one clamp, and
the clip is configured to secure a ridge provided along the longitudinal axis of the proximal end of the continuum robot.
